SEQUENCE LISTING (4 sequences)

SEQ ID NO: 1 - DNA, 56 bp, Artificial
  LNA/DNA Chimera Sense Strand Primer
gctcaggaacaaagaaacgcagggagagaggaaggaattcaccagtcacacgacca

SEQ ID NO: 2 - DNA, 56 bp, Artificial
  LNA/DNA Chimera Sense Strand Primer
cagtaacagatacaaactcaagggagagaggaaggaattcaccagtcacacgacca

SEQ ID NO: 3 - DNA, 17 bp, Artificial
  LNA/DNA - Blocking Oligo
ctccttcctctctccct

SEQ ID NO: 4 - DNA, 20 bp, Artificial
  LNA/DNA - DNA Antisense Primer
ggtgctgctatcgatggttt

(19) United States
(12) Patent Application Publication
(10) Pub. No.: US 2005/0164205 A1
(43) Pub. Date: Jul. 28, 2005

(54) Charge and mass tags for detection and analysis

(75) Inventor: Robert Steven Puskas, Manchester, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Singulex, Inc.

(21) Appl. No.: 10/720,047

(22) Filed: Nov. 19, 2003

Related U.S. Application Data
(60) Provisional application No. 60/427,232, filed on Nov. 19, 2002.
(60) Provisional application No. 60/427,233, filed on Nov. 19, 2002.
(60) Provisional application No. 60/427,234, filed on Nov. 19, 2002.

(51) Int. Cl. C12Q 1/68

(52) U.S. Cl. 435/6

(57) ABSTRACT

A method for detecting molecules, molecular interactions or molecular complexes comprising: (a) detecting, through interaction with at least one probe, an interaction between the probe and a molecule, wherein at least one probe has a differentiating physical characteristic selected from the group consisting of a net charge, a mass, and any combination thereof, and at least one probe has a detectable parameter; and (b) differentiating between an unbound probe or probes of (a) and a probe or probes bound to the molecule.

### CROSS-REFERENCE TO RELATED APPLICATIONS

This application claims priority from Provisional Application Ser. Nos. 60/427,232, 60/427,233, and 60/427,234, each filed on Nov. 19, 2002, and each of which is incorporated herein by reference in its entirety.

### STATEMENT REGARDING FEDERALLY SPONSORED RESEARCH OR DEVELOPMENT

Not Applicable.

### REFERENCE TO A SEQUENCE LISTING

The Sequence Listing, which is a part of the present disclosure, includes a text file comprising nucleotide and/or amino acid sequences of the present invention on a floppy disk. The subject matter of the Sequence Listing is incorporated herein by reference in its entirety.

### BACKGROUND OF THE INVENTION

1. Field of the Invention

The present invention relates to methods for counting, differentiating and identifying molecules, molecular interactions and molecular complexes via direct detection of interaction with one or more probes that contain charge, mass or charge and mass tags.

2. Description of the Related Art

Probe-cleavage systems. Methods have been developed to use multiple probes with a specific species of probe for each species of target. In these systems the probes are cleaved from the probe target complex after their interaction with target, each species of probe producing a specific cleavage product. The cleaved, released probes are detected and their presence indicates indirectly the presence of specific targets in the sample.

Barron (Barron, US 2003/008856) claims the use of specific polyamide-polynucleotide primer conjugates to determine the nucleotide sequence of target nucleic acid molecules. The primer conjugates are hybridized to target and the primer is extended in the presence of at least one chain terminator to form conjugated nucleic acid molecule fragments. The conjugated nucleic acid molecule fragments are separated by electrophoresis in a non-sieving matrix and the nucleotide sequence of the target is determined from the nucleotide sequence of the separated nucleic acid fragments

Barron also describes the use of uncharged, monodisperse perturbing entities (“drag tags”) to DNA molecules, creating hydrodynamic drag which is not related to DNA size. This allows separation of differently sized DNA molecules in a non sieving solution. The monodisperse drag tag is added to all nucleic acids in the sample solution. Large molecules in the sample are affected the least; small molecules in the sample are affected the most. The perturbing entities used are poloypeptoids and peptide/peptoide chamerae.

In a reversed version of this method Barron performs molar mass profiling of synthetic polymers. A monodisperse DNA “engine” is conjugated to an uncharged polydisperse polymer. The conjugates are separated electrophoretically in a non-sieving medium. Large polymer molecules are sped up less by the DNA than are small polymer molecules. The result is separation of the polymers by size.

The above methods have been proposed for use in DNA sequencing, although their utility for that purpose has not been demonstrated.

There is an unmet need for methods to use one or more specific targets that are modified prior to their use in the specific recognition of target. When two or more probes are used in the target recognition process, only one probe need have a modified charge, mass or charge mass (CM). The probe(s)-target complexes are differentiated based on their charge, mass, or charge to mass ratio after interaction of the probe(s) and target. The target may or may not be treated prior to probe-target recognition and analysis to facilitate detection, analysis or differentiation of the probe-target complex. For example target nucleic acids may be cleaved with a restriction endonuclease prior to analysis to enhance the differentiation of probe(s) target complexes.

### BRIEF SUMMARY OF THE INVENTION

Accordingly, it is an object of the invention to overcome these and other problems associated with the related art. These and other objects, features and technical advantages are achieved by providing charge mass tags and methods of using same.

Accordingly, an aspect of the invention provides a method for detecting molecules, molecular interactions or molecular complexes comprising:
(a) detecting, through interaction with at least one probe, an interaction between the probe and a molecule, wherein at least one probe has a differentiating physical characteristic selected from the group consisting of a net charge, a mass, and any combination thereof, and at least one probe has a detectable parameter; and
(b) differentiating between an unbound probe or probes of (a) and a probe or probes bound to the molecule.

Another aspect of the invention provides a method for detecting and molecules, molecular interactions or molecular complexes, consisting of:
a. One or more probes
b. Each probe recognizing (via hybridization, binding, etc.) at least one target molecule species, or site on a target molecule species (or another probe), and
c. At least one of the probes has a net charge, mass or net charge and mass (any or the three or which is designated as a CM tag) that is differentiable from the target or from one or more of the other probes, and
d. At least one probe or target that has a detectable parameter
e. Differentiating between an unbound CM probe and a probe bound to a target (complex, forming an interaction, or to another probe) based upon a temporal difference in detection between the unbound CM probe and the bound CM probe target complex in an electric field, or
f. Differentiating CM probe based upon a temporal difference in detection of either the unbound CM probe or the bound CM probe-target complex by virtue of temporal difference in detection between the detected entity's or entities' behavior in an electric field.

These and other features, aspects and advantages of the present invention will become better understood with reference to the following description, examples and appended claims.

### BRIEF DESCRIPTION OF THE SEVERAL VIEWS OF THE DRAWINGS

FIG. 1.

FIG. 2.

FIG. 3.

FIG. 4.

FIG. 5

FIG. 6.

### DETAILED DESCRIPTION OF THE INVENTION

Abbreviations and Definitions

To facilitate understanding of the invention, a number of terms and abbreviations as used herein are defined below as follows:

To facilitate understanding of the invention, a number of terms and abbreviations as used herein are defined below as follows:

The term “label” refers to a moiety that, when attached to the compositions of the invention, render such compositions detectable using known detection means, e.g., spectroscopic, photochemical, radioactive, biochemical, immunochemical, enzymatic or chemical means. Exemplary labels include but are not limited to fluorophores, chromophores, radioisotopes, spin labels, enzyme labels and chemiluminescent labels. Such labels allow direct detection of labeled compounds by a suitable detector, e.g., a fluorescence detector. In addition, such labels include components of multi-component labeling schemes, e.g., a system in which a ligand binds specifically and with high affinity to a detectable anti-ligand, e.g., a labeled antibody binds to its corresponding antigen.

“Linking group” means a moiety capable of reacting with a “complementary functionality” to form a “linkage.” A linking group and its associated complementary functionality is referred to herein as a “linkage pair.” Preferred linkage pairs include a first member selected from the group isothiocyanate, sulfonyl chloride, 4,6-dichlorotriazinyl, succinimidyl ester, or other active carboxylate, and a second member that is amine. Preferably a first member of a linkage pair is maleimide, halo acetyl, or iodoacetamide whenever the second member of the linkage pair is sulfhydryl. (e.g., R. Haugland, Molecular Probes Handbook of Fluorescent Probes and Research Chemicals, Molecular probes, Inc. (1992)). In a particularly preferred embodiment, the first member of a linkage pair is N-hydroxysuccinimidyl (NHS) ester and the second member of the linkage pair is amine, where, to form an NHS ester, a carboxylate moiety is reacted with dicyclohexylcarbodiimide and N-hydroxysuccinimide.

The term “Watson/Crick base-pairing” refers to a pattern of specific pairs of nucleotides, and analogs thereof, that bind together through sequence-specific hydrogen-bonds, e.g. A pairs with T and U, and G pairs with C.

The term “nucleoside” refers to a compound comprising a purine, deazapurine, or pyrimidine nucleobase, e.g., adenine, guanine, cytosine, uracil, thymine, 7-deazaadenine, 7-deazaguanosine, and the like, that is linked to a pentose at the 1′-position. When the nucleoside base is purine or 7-deazapurine, the pentose is attached to the nucleobase at the 9-position of the purine or deazapurine, and when the nucleobase is pyrimidine, the pentose is attached to the nucleobase at the 1-position of the pyrimidine, (e.g., Komberg and Baker, DNA Replication, 2nd Ed. (Freeman, San Francisco, 1992)). The term “nucleotide” as used herein refers to a phosphate ester of a nucleoside, e.g., a triphosphate ester, wherein the most common site of esterification is the hydroxyl group attached to the C-5 position of the pentose. The term “nucleoside/tide” as used herein refers to a set of compounds including both nucleosides and nucleotides.

The term “polynucleotide” means polymers of nucleotide monomers, including analogs of such polymers, including double and single stranded deoxyribonucleotides, ribonucleotides, .alpha.-anomeric forms thereof, and the like. Monomers are linked by “internucleotide linkages,” e.g., phosphodiester linkages, where as used herein, the term “phosphodiester linkage” refers to phosphodiester bonds or bonds including phosphate analogs thereof, including associated counterions, e.g., H.sup.+, NH.sub.4.sup.+, Na.sup.+, if such counterions are present. Whenever a polynucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxyguanosine, and “T” denotes deoxythymidine, unless otherwise noted.

“Analogs” in reference to nucleosides/tides and/or polynucleotides comprise synthetic analogs having modified nucleobase portions, modified pentose portions and/or modified phosphate portions, and, in the case of polynucleotides, modified internucleotide linkages, as described generally elsewhere (e.g., Scheit, Nucleotide Analogs (John Wiley, New York, (1980); Englisch, Angew. Chem. Int. Ed. Engl. 30:613-29 (1991); Agrawal, Protocols for Polynucleotides and Analogs, Humana Press (1994)). Generally, modified phosphate portions comprise analogs of phosphate wherein the phosphorous atom is in the +5 oxidation state and one or more of the oxygen atoms is replaced with a non-oxygen moiety, e.g., sulfur. Exemplary phosphate analogs include but are not limited to phosphorothioate, phosphorodithioate, phosphoroselenoate, phosphorodiselenoate, phosphoroanilothioate, phosphoranilidate, phosphoramidate, boronophosphates, including associated counterions, e.g., H.sup.+, NH.sub.4.sup.+, Na.sup.+, if such counterions are present. Exemplary modified nucleobase portions include but are not limited to 2,6-diaminopurine, hypoxanthine, pseudouridine, C-5-propyne, isocytosine, isoguanine, 2-thiopyrimidine, and other like analogs. Particularly preferred nucleobase analogs are iso-C and iso-G nucleobase analogs available from Sulfonics, Inc., Alachua, Fla. (e.g., Benner, et al., U.S. Pat. No. 5,432,272). Exemplary modified pentose portions include but are not limited to 2′-or 3′-modifications where the 2′-or 3′-position is hydrogen, hydroxy, alkoxy, e.g., methoxy, ethoxy, allyloxy, isopropoxy, butoxy, isobutoxy and phenoxy, azido, amino or alkylamino, fluoro, chloro, bromo and the like. Modified internucleotide linkages include phosphate analogs, analogs having achiral and uncharged intersubunit linkages (e.g., Sterchak, E. P., et al., Organic Chem, 52:4202 (1987)), and uncharged morpholino-based polymers having achiral intersubunit linkages (e.g., U.S. Pat. No. 5,034,506). A particularly preferred class of polynucleotide analogs where a conventional sugar and internucleotide linkage has been replaced with a 2-aminoethylglycine amide backbone polymer is peptide nucleic acid (PNA) (e.g., Nielsen etal., Science, 254:1497-1500 (1991); Egholm etal., J. Am. Chem. Soc., 114: 1895-1897 (1992)).

As used herein the term “primer-extension reagent” means a reagent comprising components necessary to effect an enzymatic template-mediated extension of a polynucleotide primer. Primer extension reagents include (1) a polymerase enzyme, e.g., a thermostable DNA polymerase enzyme such as Taq polymerase; (2) a buffer; (3) one or more chain-extension nucleotides, e.g., deoxynucleotide triphosphates, e.g., deoxyguanosine 5′-triphosphate, 7-deazadeoxyguanosine 5′-triphosphate, deoxyadenosine 5′-triphosphate, deoxythymidine 5′-triphosphate, deoxycytidine 5′-triphosphate; and, optionally in the case of Sanger-type DNA sequencing reactions, (4) one or more chain-terminating nucleotides, e.g., dideoxynucleotide triphosphates, e.g., dideoxyguanosine 5′-triphosphate, 7-deazadideoxyguanosine 5′-triphosphate, dideoxyadenosine 5′-triphosphate, dideoxythymidine 5′-triphosphate, and dideoxycytidine 5′-triphosphate.

“Mobility-dependent analysis technique” means an analysis technique based on differential rates of migration between different analyte species. Exemplary mobility-dependent analysis techniques include electrophoresis, chromatography, sedimentation, e.g., gradient centrifugation, field-flow fractionation, multi-stage extraction techniques and the like.

A “target nucleic acid” sequence for use with the present invention may be derived from any living, or once living, organism, including but not limited to prokaryote, eukaryote, plant, animal, and virus. The target nucleic acid sequence may originate from a nucleus of a cell, e.g., genomic DNA, or may be extranuclear nucleic acid, e.g., plasmid, mitrochondrial nucleic acid, various RNAs, and the like. The target nucleic acid sequence may be first reverse-transcribed into cDNA if the target nucleic acid is RNA. Furthermore, the target nucleic acid sequence may be present in a double stranded or single stranded form.

A “probe” refers to a biopolymer comprising a recognition (R) moiety and tail (T) moiety of the general form R-B-T where B is a bridge connecting the recognition and tail moieties. A labeled probe further comprises one or more detectable label moieties covalenty or non-covalently attached to or incorporated in the tail. The recognition moiety is a nucleic acid sequence which complementary to a nucleic acid sequence in the target molecule. The tail moiety is a defined length polynucleotide which may or may not include labels and/or mass tags.

Parameter unit. A measure of a parameter which is discernable above the background measurement level of the analytical system, and which is associated with a specific, unitized probe (molecule), and which is designated as the base unit of measurement for the specific, unitized probe for that parameter. A probe may possess more than one type of parameter unit. A probe may have parameter units or subunits for more than one parameter. Measurement of more than one parameter may be used to discern (identify) a specific unitized probe or probe target. Probes detected simultaneously (by measurement of a parameter) can be discriminated from one another (or by inference associated with the target molecule) by measuring one or more other parameters (parameter units) possessed by one or more of the detected probes. A parameter of the target also may be used to distinguish between probe-targets and probes. (The parameter unit for a probe also may be tied to a given set of conditions, e.g. velocity in our instrument system).

It is of interest to maximize the detection and differentiation of analytes for increased sensitivity. The ultimate detection level is that of detecting individual molecules, interactions between molecules and molecular complexes. The following invention details methods for detecting such molecules, molecular interactions or molecular complexes by means of probes with charge-mass tags to be used in single molecule analysis.

Single molecule electrophoresis involves measuring the electrokinetic velocity of molecules (combined electrophoretic velocity and electro-osmotic force) in solutions in a transport tube. Currently target molecules can be detected and differentiated based on their luminescence parameters, charge, mass and shape. The present invention involves means to affect detectable parameters and the mass or charge of target molecules so as to make them detectable and to enhance their differentiation in single molecule analysis (and especially single molecule electrophoresis).

Probes useful in this invention will have two properties—a detectable parameter and/or a charge, mass or charge and mass tag (a CM tag) sufficient to affect the velocity of probe-target complex in the analytical system used. Most probes will posses a detectable parameter and a CM tag, but probes do not need to posses both properties to be useful in single molecule analysis. In fact probes with CM tags alone can be used if combined with one or more probes that possess at least one detectable parameter, or if they interact with a target that possesses a detectable parameter. Probes may be a nucleic acid, oligonucleotide, peptide, polypeptide, lipid, sterol, biological molecule, dye, drug, mass tag, isotope, and any combination thereof.

A commonly used detectable parameter is luminescence. Not all target molecules are luminescent, but detectable luminescent probes that can interact with target molecules can made by those skilled in the art. Luminescent detectable parameters include fluorescence lifetime, fluorescence polarization, fluorescence, luminescence, burst size, burst length, chemiluminescence, light absorption, and other parameters known to those experienced in the art.

Additional detectable parameters of probes useful in this invention include, but are not limited to electrical reactance, enzyme activity, diffusion, cooperative hybridization and other non-luminescent-related parameters known to those experienced in the art.

A CM tag also may be a nucleic acid, oligonucleotide, peptide, polypeptide, lipid, sterol, biological molecule, dye, drug, isotope, as well as a microsphere, nanosphere, barcode particle, or nanocrystal. Probes themselves or probe components themselves may act as CM tags. Probe components with detectable parameters also can serve as CM tags.

The invention involves the use of at least one probe with one or more CM tags and at least one (even the same) probe with at least one detectable parameter to specifically detect a target and make it differentiable from other targets. A single CM tag probe specific for a target also will make its target differentiable if the target itself possess a detectable parameter.

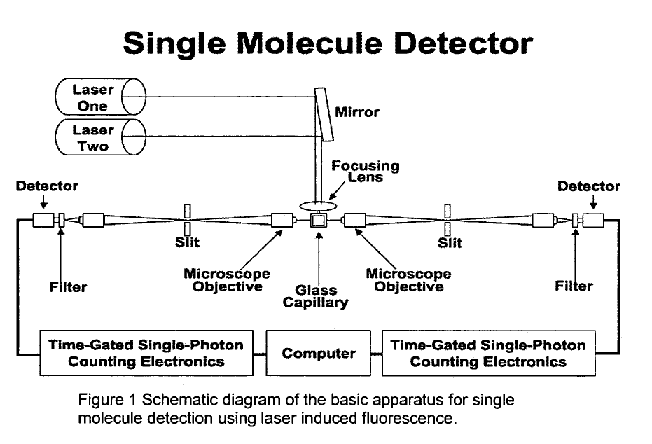

Detection may be by means of optical or electrical detection and may involve spatial or temporal parameters for detection. A method and various potential combinations of luminescent probes and CM tag probes for detection of interaction with target are shown in FIGS. 1-4. The method and combinations are not shown by way of limitation, but merely as examples of the use of CM tags.

FIG. 1 shows a diagram of the method for single molecule electrophoresis. Two laser beams pass through a rectangular capillary tube. The capillary tube is filled with sample and current is applied to the sample. Fluorescent molecules in the sample move through each laser beam in response to the applied electric field. Laser induced fluorescence occurs as each fluorescent molecule passes through one of the laser beams. Fluorescence burst events are crosscorrelated to indicate the time for passage of molecules between the two laser beams (indicative of their velocity in the system). Each molecules velocity is dependent upon its charge, mass and size. Because nucleic acids generally have a fixed charge/mass ratio per unit length, often sieving material will be added to samples in this system to enhance velocity differences between nucleic acid molecules of different sizes.

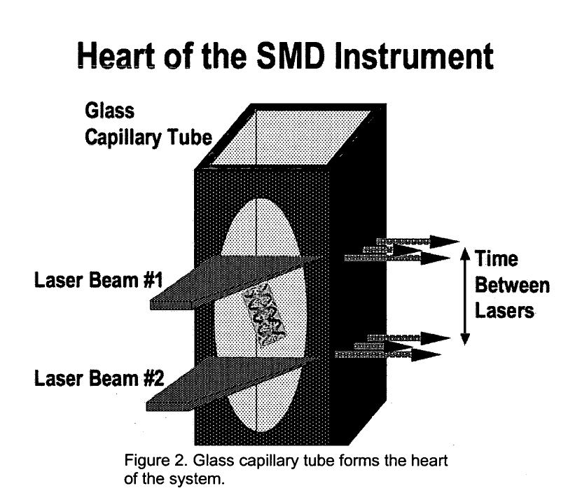

In FIG. 2 Probes A and B are fluorescent and also act as CM tags by virtue of the dye molecule(s) conjugated to the probe. The symbol (#) in all figures corresponds to the number of crosscorrelated fluorescent events detected at a specific velocity. The CM tag value for probe A is less than that for Probe B. Differentiation between single and double hybrids to target can be made based on fluorescence, fluorescence intensity and velocity in an electric field. Any two of the mentioned parameters can be used to identify a particular target-probe combination. Alternatively, the target also may be labeled with one or more dyes or CM tags to facilitate identification.

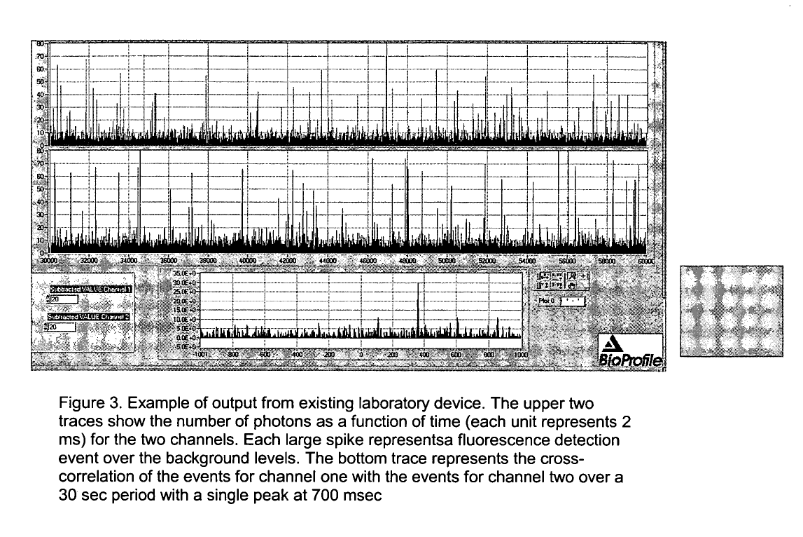

In FIG. 3 Fluorescence Intensity and mass tags are used to identify pairs of probes that have hybridized to different target molecules. All probes are fluorescent and some incorporate mass tags. Probes A and B are detected based merely on an increase in fluorescence intensity per detection time unit. This method of detection is claimed in the 2002 Provisional Patent submitted by BioProfile (Detection of Target Molecules Through Interaction with Probes). Probes C and D combine increased intensity from simultaneous hybridization of probes B and C and the change in velocity from the mass tag (m) on probe B as means to detect the B-C-target hybrid. Probes E and F both affect intensity and mass of the hybridization complex for differentiation and detection. Probes G and H are similar to probes C and D, but probe H incorporates a larger mass tag. Probes I and J are similar to probes E and F, but have incorporated larger mass tags.

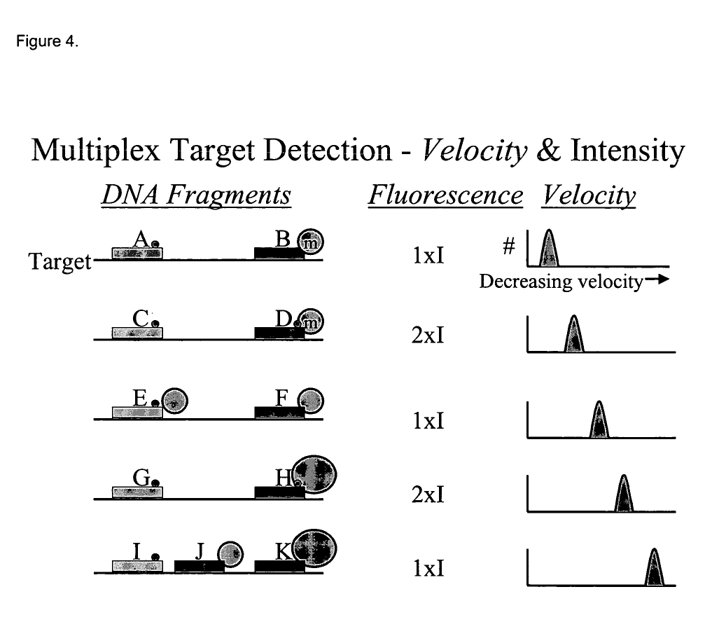

In FIG. 4 some probes are fluorescent, while others are not. Some probes incorporate mass tags while others do not. Some mass tags are fluorescent while others are not. It can be seen that only one probe (or the target) need be fluorescent to detect multiple targets or to detect hybridization of multiple probes to a target and identify which probes have hybridized to the target. Two or more probes with mass tags can be used with one or more fluorescent probes as shown with probes I, J & K.

The above examples show nucleic acid interactions (hybridization's), but of course the probes may be of other types of molecules and combinations thereof as outlined in this document.

Single molecule detection is the ultimate limit for detection. Within the field of single molecule detection, optical-based detection systems (i.e. laser-induced fluorescence) are generally considered the most desirable option currently available for detection of biochemicals. Optical microscopy was used in the first published documentation of single molecule detection (Hirschfeld 1976). Since then, the field of optical detection of single molecules has been rapidly advanced by a variety of improvements in methodology.

A typical system used for detection in fluids involves a fluid flow system similar to flow cytometry systems in which fluorescence from a sample stream is measured as the stream passes a detector. These methods have been applied for sizing of DNA fragments (Ambrose et al. 1993; Castro et al. 1993; Goodwin et al. 1993a; Huang et al. 1996; Johnson et al. 1993; Larsen et al. 2000; Petty et al. 1995), for progress toward single molecule DNA sequencing (Ambrose et al. 1993; Davis et al. 1991; Goodwin et al. 1995; Goodwin et al. 1996; Goodwin et al. 1993b; Harding and Keller 1992; Jett et al. 1989; Soper et al. 1991a; Van Orden et al. 2000; Werner et al. 2003), and to detect DNA hybridization via two-color fluorescence (Castro and Williams 1997). A variation of the fluid flow technology involves application of an electric field in which molecules move electrophoretically (Castro and Al. 1995; Castro and Shera 1995; Chen and Dovichi 1996; Haab and Mathies 1995; Ma et al. 2001; Shortreed et al. 2000; Soper et al. 1995b; Van Orden and Keller 1998)and which has been utilized for sizing of DNA. Electrophoretic single molecule analysis (molecular electrophoresis) also has been used to detect protein-DNA interactions via two-color fluorescence (LeCaptain et al. 2001). To date no system has combined the differentiating capabilities and single molecule sensitivity of molecular electrophoresis for multiplexed hybridization detection.

Systems capable of single-molecule detection use a single laser or laser beam to detect down to single molecules on surfaces or in solution. Fluorescence correlation spectroscopy and other confocal instrumentation interrogate minute volumes, consequently have limited molecular throughput, and are not suitable for detecting very low levels of analytes (Nie and Zare 1997). Atomic Force Microscopy can detect single molecules, but it also suffers from limited molecular throughput (Wrotnowski 2002). These systems also have limited potential for multiplexing. Our single molecule electrophoresis instrument offers single molecule detection, high specificity, high molecular throughput and extensive multiplexing capabilities.

Single Molecule Detection System

Our basic method for ultrasensitive detection relies on single-molecule fluorescence analysis. A sample to be analyzed is first obtained from air, water, surfaces or tissue. No polymerases, enzymes or proteins, or any amplification processes are necessary so sample preparation times and complexity are minimal. This single molecule detection instrument provides an ultrasensitive means to reliably detect individual molecules.

The basic detection scheme is shown in FIG. 1. The apparatus consists of two lasers (or a single laser source split into two beams), focusing light-collection optics, two single photon detectors, and detection electronics under computer control. A sample compartment (not shown) contains two reservoirs that hold the solution being analyzed. The reservoirs are connected by tubing to a glass capillary cell that is square in cross section.

The heart of the system is the glass capillary flow cell of the apparatus shown in FIG. 2. Two laser beams (5 μm in diameter) are optically focused about 100 μm apart perpendicular to the length of the sample-filled capillary tube. The lasers are operated at particular wavelengths depending upon the nature of the detection probe to be excited. The interrogation volume of the detection system is determined by the diameter of the laser beam and by the segment of the laser beam selected by the optics that direct light to the detectors. The interrogation volume is set such that, with an appropriate sample concentration, single molecules (single nucleic acid probes or single probe-target hybrids) are present in the interrogation volume during each time interval over which observations are made.

Molecules are pumped through the capillary, or an electric field is applied to the sample to move molecules electrophoretically. Under electrophoretic conditions, like molecules move through the tube in lockstep (plug flow). As molecules pass through each laser beam, excitation of each fluorescent molecule takes place via one-photon excitation. Within a fraction of a second, the excited molecule relaxes, emitting a detectable burst of light. The excitation-emission cycle is repeated many times by each molecule in the length of time it takes for it to pass through the laser beam allowing the instrument to be able to detect hundreds of molecules per second. Photons emitted by fluorescent molecules are registered in both detectors with a time delay indicative of the time for the molecule (or molecular hybridization complex) to pass from the interrogation volume of one detector to the interrogation volume of the second detector. The photon intensity recorded by the detectors is then cross-correlated using a personal computer with a digital correlator card. The computer then produces a histogram of velocities that shows a peak for every fluorescent species present in the sample. When the sample is pumped through the capillary, all molecules move at the same velocity. When an electric field is applied to the sample, the transit time between the detectors for each molecule is dependent upon the molecule's characteristic charge, size and shape (Castro and Al. 1995; Castro and Shera 1995; Castro and Williams 1997). An example of the output of the existing laboratory system is shown in FIG. 3.

With the current instrument configuration (5 μm laser beam) approximately 0.25% of the fluorescent molecules in the solution pass through the laser beams and are detectable. This percentage can be increased by configuring each laser beam such that it forms a narrow band perpendicular to the length of the capillary (as shown in FIG. 2). Such an arrangement can raise the percentage of detectable molecules to approximately 5% of the molecules in the solution. Other configurations illuminating larger areas of the capillary have been calculated to enable detection of up to 50% of the fluorescent molecules present in a sample.

Methods of Use

The present invention further encompasses methods of using probes, as well as compositions comprising a plurality of probes, and wherein each probe has a distinctive ratio of charge to translational frictional drag, to detect and characterize one or more selected nucleotide sequences within one or more target nucleic acids.

In one aspect, the present invention provides a method of detecting a plurality of sequences within one or more target nucleic acids, comprising contacting a plurality of, wherein each probe has a structure independently, with one or more target nucleic acids, generally under conditions that distinguish those that hybridize to the target nucleic acid, and detecting those which have hybridized to the target nucleic acid.

In one aspect of this method, the target nucleic acids are immobilized. In this aspect, the immobilized target nucleic acids are contacted with sequence-specific probes, which further comprise a detectable label, under conditions that distinguish those probes having sufficient homology to hybridize to the target nucleic acid. The non-hybridized probes are washed away and hybridized probes are recovered and detected after denaturation of the base-paired structure formed between the sequence-specific probe and the immobilized target nucleic acid.

In another aspect of this method, the target nucleic acid, which may be immobilized, is contacted with a plurality of sequence-specific probe probes whereby two probes hybridize to adjacent sequences of the target nucleic acid such that the 5′-end of one probe, which generally will carry a 5′-phosphate moiety, abuts the 3′-end of the second probe, so that the two probes can be covalently joined to one another, in certain embodiments, with a DNA chemical or enzymatic ligating activity, to form a ligated product. In this aspect of the method, the ligated product is formed by the joining of two probes, at least one of which comprises a detectable label and at least one of which is a sequence-specific probe, such that the ligated product has a distinctive ratio of charge to translational frictional drag. In a further aspect, three or more probes are hybridized to adjacent sequences of a target nucleic acid in such a manner that at least three probes can be covalently joined to form a ligated product, wherein at least one of the probes so joined comprises a detectable label, and at one of the probes so joined is a sequence-specific such that the ligated product has a distinctive ratio of charge to translational frictional drag. Generally, the ligated product, which is hybridized to the target nucleic acid, is released by denaturation, and the ligated product having a distinctive ratio of charge to translational frictional drag, is detected and analyzed, to provide information about the selected nucleotide sequence within the target nucleic acid.

This cycle of hybridization, joining, and denaturation, may be repeated in order to amplify the concentration of the ligated product formed. In this instance, the joining is optionally accomplished by means of a thermostable ligating enzyme. These reactions are conveniently carried out in thermal cycling machines with thermally stable ligases.

Furthermore, additional probes, which together are sufficiently complementary to the ligated product to hybridize thereto and be covalently joined to one another as above, are also included, thereby affording geometric amplification of the ligated product, i. e., a ligase chain reaction (Wu, D. Y. and Wallace B. (1989), The ligation amplification reaction (LAR)-Amplification of Specific DNA sequences using sequential Rounds of Template Dependent Ligation, Genomics 4:560-569; Barany, (1991), Proc. Natl. Acad. Sci. USA, 88:189; Barany, (1991), PCR Methods and Applic., 1:5). To suppress unwanted ligation of blunted ended hybrids formed between complementary pairs of the and second oligonucleotides and the second pair of oligonucleotides, conditions and agents inhibiting blunted ended ligation, for example 200 mM NaCI and phosphate, are included in the ligation reaction.

The product of such a ligase chain reaction therefore is a double stranded molecule consisting of two strands, each of which is the product of the joining of at least two sequence-specific probes. Accordingly, in yet another aspect of the present invention, at least one of the incorporated within the ligase chain reaction product comprises a detectable label, and at one of the is a sequence-specific probe such that the ligase chain reaction product has a distinctive ratio of charge to translational frictional drag.

In another aspect of the oligonucleotide ligase assays described above, mismatches, i.e. non-complementary nucleobases, existing between selected nucleotide sequences within the target nucleic acid and either or both of the sequence-specific probe and the second oligonucleotide interfere with the ligation of the two probes either by preventing hybrid formation or preventing proper joining of the adjacent terminal nucleotide residues. Thus, when the binding conditions are chosen to permit hybridization of both probes despite at least one mismatch, the formation of a ligated product reveals the sequence of the selected nucleotide sequence as it exists within the target nucleic acid, at least with respect to the terminal, adjacent residues of the two probes. Those skilled in the art are well versed in selecting appropriate binding conditions. such as cation concentration, temperature, pH, and oligonucleotide composition to selectively hybridize the probes to the selected nucleotide sequences within the target nucleic acid.

Since the base pairing of terminal adjacent residues affects ligation, in one embodiment the probe providing the 3′ terminal nucleobase involved in the joining reaction is designed to be perfectly complementary to the target sequence while the probe providing the 5′ terminal nucleobase residue involved in the joining reaction is designed to be perfectly complementary in all but the 5′ terminal nucleobase. In another embodiment, the probes are designed such that the probe providing the 3′ terminal nucleobase is perfectly complementary except for the 3′ terminal nucleobase residue while the oligonucleotide providing the 5′ terminal nucleobase is perfectly complementary (Wu, D. Y. and Wallace, B.,(1989), Specificity of nick-closing activity of bacteriophage T4 DNA ligase, Gene 76: 245-254; Landegren, U. et al. (1988) A ligase mediated gene detection technique, Science 2241: 1077-1080).

In a modification of the method set forth above, the sequence-specific probe comprises a nucleobase sequence that is complementary to the target sequence, but comprises a non-terminal mismatch with respect to non-target sequences. In this aspect of the invention, the composition of the sequence-specific probe and the nature of the experimental conditions are such that the probe will only hybridize to the target sequence. In this embodiment for example, a second probe that hybridizes to the target nucleobase, either upstream or downstream of the hybridized sequence-specific probe, may be ligated to that probe to form the ligated, product that is diagnostic of the presence of the target nucleotide sequence.

In a further modification of the embodiment set forth above, the sequence-specific probe is hybridized to a selected nucleotide sequence within a target nucleic acid that is immediately adjacent to the site of interest. A second sequence-specific probe is hybridized to the selected region within the target nucleic acid such that the hybridized oligonucleotides are separated by a gap of at least one nucleotide residue. In another embodiment, the length of the gap is a single nucleotide residue representing a single polynucleotide polymorphism in the target nucleic acid. Following hybridization, the complex, which consists of the two probes hybridized to the target nucleic acid, is treated with a nucleic acid polymerase in the presence of at least one deoxyribonucleoside triphosphate. If the deoxyribonucleoside triphosphate(s) provided are complementary to the target polynucleotide's nucleotide residues which define the gap, the polymerase fills the gap between the two hybridized probes. Subsequent treatment with ligase joins the two hybridized oligonucleotides to form a ligated, product, which can, in one embodiment, be separated from the template by thermal dissociation, thereby providing a diagnostic product having a distinctive ratio of charge to translational frictional drag. This diagnostic product will generally comprise a reporter molecule, which may be included within either of the ligated probes, be attached to the one or more nucleobases added by the polymerizing activity, or be added subsequent to the covalent joining of the probes. By treating with polymerase in the presence of fewer than four nucleoside triphosphates, the nucleotide residues comprising the gap may be determined. Further amplification of ligated product is achieved by repeated cycles of denaturation, annealing, nucleic acid polymerase gap filling, and ligation in the presence of at least one of the nucleoside triphosphates.

If the treatment with nucleic acid polymerase occurs in the presence of one labeled nucleoside triphosphate or a mixture containing one labeled and 3 unlabeled nucleoside triphosphates, ligated products comprising at least one incorporated, labeled nucleoside are readily detected upon electrophoretic separation of the labeled ligated products. Modification of the nucleotide mixture to one having one labeled nucleoside triphosphate and three chain terminating nucleoside triphosphates suppresses unwanted ligation of oligonucleotides with incorrectly incorporated nucleotide residues.

Probes of the present invention are also useful as primers for nucleic acid sequence analysis by the chain termination method, a method well known to those skilled in the art. In one embodiment, probes are hybridized to target nucleic acid and extended by a nucleic acid polymerase in the presence of a mixture of nucleoside triphosphates and a chain terminating nucleoside triphosphate. The polymerase reaction generates a plurality of chain terminated nucleic acids fragments, which are separated, for example by capillary electrophoresis. Chain termination by the incorporated chain terminating nucleoside triphosphate identifies the 3′ terminal residue of the terminated nucleic acid fragment.

For the purposes of detecting the chain terminated species, various substituents of the nucleic acid fragments are amenable to conjugation with detectable reporter molecules. These include the nucleoside triphosphate precursors, including the chain terminators, incorporated into the nucleic acid. Detectable reporter molecules may be radioactive, chemiluminescent, bioluminescent, fluorescent, or ligand molecules. In one embodiment, the detectable label is a fluorescent molecule, for example fluorescein isothiocyanate, Texas red, rhodamine, and cyanine dyes and derivatives thereof. In another embodiment, the fluorescent dyes are to reduce the variations in electrophoretic mobility of nucleic acids caused by the fluorescent label (see e.g. Ju, J. et al. (1995). Design and synthesis of fluorescence energy transfer dye-labeled primers and their application for DNA sequencing and analysis, Anal. Biochem. 231: 131-40; Metzker, et al. (1996) Electrophoretically uniform fluorescent dyes for automated DNA sequencing, Science 271: 1420-22; Hung, S. C. et al. (1997) Comparison of fluorescence energy transfer primers with different donor-acceptor dye combinations, Anal Biochem. 252: 77-88; Tu, 0. et al. (1998) The influence of fluorescent dye structure on the electrophoretic mobility of end-labeled DNA, Nucleic Acids Res. 26: 2797-2802)

In one embodiment, the of the present invention are used within a format for sequencing selected regions within a target polynucleotide wherein one of four spectrally resolvable fluorescent molecules is used to label the nucleic acid fragments in reactions having one of four chain terminating nucleoside triphosphates. In another aspect of this embodiment, the probes of the present invention are used for sequencing selected regions within a target polynucleotide wherein one of four spectrally resolvable fluorescent molecules is used to label an oligonucleotide primer in a reaction containing one of four chain terminating nucleoside triphosphates. Thus, in both aspects of this embodiment, detecting the fluorescent color of the chain terminated nucleic acid fragment identifies the 3′ terminal nucleotide residue. Separation of the chain-terminated products by electrophoresis, typically in a single gel lane or capillary, along with simultaneous on-line detection of four spectrally resolvable fluorescent molecules allows rapid sequence determination from the colors of the separated nucleic acid fragments (Prober, J. M. et al. (1985), A System for Rapid DNA Sequencing with Fluorescent Chain Terminating Dideoxynucleotides, Science 238: 336-341; Karger, A. E. et al., (1991), Multiwavelength Fluorescence Detection for DNA Sequencing Using Capillary Electrophoresis, Nucleic Acids Res. 19 (18):4955-62).

When the nucleotide sequence of interest is a small region of the target nucleic acid, for example a site including single nucleotide polymorphism, modified sequencing formats, optionally, are used. In one such embodiment, a sequence-specific probe is hybridized in a sequence-specific manner such that the 3′-terminal nucleotide residue of the sequence-specific probe is immediately adjacent to the site of interest. The hybridized probe is extended by a nucleic acid polymerase in the presence of at least one chain terminating nucleoside triphosphate extends the oligonucleotide by one nucleotide if the chain terminating nucleotide is complementary to the target nucleic residue immediately downstream of the 3′-terminus of the hybridized sequence-specific probe. Separation and detection of the extended, sequence-specific sequence-specific probe provides the identity of the residue immediately adjacent to the hybridized primer. In this embodiment, the use of a plurality of different probe permits the simultaneous detection and analysis of a plurality of target sequences in a single separation.

Detecting the extended primer is accomplished by including a reporter molecule conjugated to the extended, sequence-specific probes are used as primers in the same manner as described above for standard sequencing reactions. Thus, in one embodiment, the chain terminating nucleoside triphosphate is labeled with one of four spectrally resolvable fluorescent molecules such that the fluorescent label uniquely identifies the chain terminating nucleotide. The composition of the residue immediately adjacent to the hybridized oligonucleotide primer is then readily ascertained from the colors of the extended oligonucleotide primer. As will be apparent to those skilled in the art, this modified sequencing format is adaptable to other mixtures of fluorescently labeled chain terminating nucleoside triphosphates. Thus the embodiments encompass nucleotide combinations having two or four chain terminating nucleoside triphosphates wherein only one chain terminator is labeled with one of four resolvable reporter labels. Mixing the products of the extension reactions, followed by separation and detection of the extended products in a single gel lane or capillary provides the ability to determine all possible sequence variations at the nucleotide residue adjacent to the hybridized primer. Further increase in sensitivity of the methods are possible by using substantially exonuclease-resistant chain terminators, such as those which form thio-ester internucleotide linkages, to reduce removal of incorporated chain terminators by polymerase associated exonuclease.

In another embodiment, are used in polymerase chain reactions (PCR) to detect and amplify selected nucleotides within one or more target nucleic acids (Mullis, K., U.S. Pat. No. 4,683,202; Saiki, R. K., et al., Enzymatic Amplification of .beta.-Globin Genomic Sequences and Restriction Site Analysis for Diagnosis of Sickle Cell Anemia, In PCR: A practical approach, M. J. McPherson, P. Quirke, and G. R. Taylor, Eds., Oxford University Press, 1991). In this aspect of the present invention, the detection method involves PCR amplification of nucleotide sequences within the target nucleic acid. In this aspect, a target nucleic acid, which may be immobilized, is contacted with a plurality of, two of which hybridize to complementary strands, and at opposite ends, of a nucleotide sequence within the target nucleic acid. Repeated cycles of extension of the hybridized sequence-specific oligonucleotides, optionally by a thermo-tolerant polymerase, thermal denaturation and dissociation of the extended product, and annealing, provide a geometric expansion of the region bracketed by the two probes. The product of such a polymerase chain reaction therefore is a double-stranded molecule consisting of two strands, each of which comprises a sequence-specific probe. In this aspect of the present invention, at least one of the sequence-specific oligonucleotides is a sequence-specific probe such that the double stranded polymerase chain reaction product has a distinctive ratio of charge to translational frictional drag. The polymerase chain reaction product formed in this aspect of the invention further comprises a label, which may be incorporated within either of the sequence-specific probes used as primers, or it may be incorporated within the substrate deoxyribonucleoside triphosphates used by the polymerizing enzyme. In yet another aspect, the polymerase chain reaction product formed is analyzed under denaturing conditions, providing separated single stranded products. In this aspect, at least one of the single stranded products comprises both a label and a sequence-specific primer such that the single-stranded product derived from double stranded polymerase chain reaction product has a distinctive ratio of charge to translational frictional drag. As is well known in the art, such a single-stranded product may also be generated by carrying out the PCR reaction with limiting amounts of one of the two sequence-specific probes used as a primer. By using distinctive sequence-specific nucleic acids or probes as primers, the PCR reaction can detect many selected regions within one or more target polynucleotides in a single assay by allowing separation of one PCR product from another. Moreover, those skilled in the art will recognize that using various combinations of primers provides additional ways to generate distinctive PCR products. For example, a combination of a probe and a second primer pair in the PCR reaction generates a PCR product with a single strand. On the other hand, a combination of a probe and a second probe, which is also mobility-modified, generates a PCR product having both strands that are mobility-modified, thus distinguishing itself from the PCR product with one strand. Thus, by varying the type of mobility-modifying group and the nucleic acid strands that are mobility-modified, the embodiments enlarge the capacity to detect multiple target segments.

Detection of the PCR products may be accomplished either during electrophoretic separation or after an electrophoretic separation. Intercalating dyes such as ethidium bromide, ethidium bromide dimers, SYBR.RTM. Green, or cyanine dye dimers such as TOTO, YOYO and BOBO are available for post separation detection (Haugland, R. P. Handbook of Fluorescent Probes and Research Chemicals, 6th ed, Molecular Probes, Inc., 1996). Alternatively, the PCR products further comprise reporter molecules, including but not limited to radioactive, chemiluminescent, bioluminescent, fluorescent, or ligand molecules that permit detection either during or subsequent to an electrophoretic separation. Methods for labeling the PCR products follow the general schemes presented for labeling in other methods described infra.

Detecting a selected nucleotide sequence within a target nucleic acid by PCR amplification also encompasses identifying sequence variations within segments of the target nucleic acid. These variations include, among others, single nucleotide polymorphisms and polymorphisms in variable nucleotide tandem repeats (VNTR) and short tandem repeats (STR), such as those defined by sequence tag sites (STS). Identifying polymorphic loci are of particular interest because they are often genetic markers for disease susceptibility (see e.g. Gastier, J. M., (1995), Hum Mol Genet, 4(10):1829-36; Kimpton, C. P., (1993), Automated DNA profiling employing multiplex amplification of short tandem repeat loci, PCR Methods Appl., 3(1): 13-22). If the polymorphisms relate to variations in VNTR or STR sequences, direct analysis of PCR products without further treatment suffices for detecting polymorphisms since the products differ in nucleotide length. The presence of PCR products, however, expands the capability of the PCR analysis to detect multiple polymorphic loci in a single reaction.

If the polymorphisms relate to single nucleotide differences, the variations are detectable by conducting PCR reactions using primers designed to have mismatches with the selected nucleic acid sequence within a target nucleic acid. The presence of intentional mismatches within the duplex formed by hybridization of the primer and the selected nucleic acid sequence within the target nucleic acid affects the thermal stability of those duplex molecules, which is reflected in the T.sub.m of those structures and thus, under selected conditions, results in preferential amplification of one target segment as compared to another. Such allele-specific polymerase chain reactions permit identification of mutations in single cells, or tissues containing a low copy number of one selected nucleotide sequences amongst a high background of other nucleotide seqeunces within one or more target nucleic acids (Cha, R. S., (1993), Mismatch amplification mutation assay (MAMA): application to the c-H-ras gene., PCR Methods Appl., 2(1) 14-20; Glaab., W. E. et al., (1999), A novel assay for allelic discrimination that combines fluorogenic 5′ nuclease polymerase chain (TaqMan.RTM.) and mismatch amplification mutation., Mutat. Res. 430: 1-12).

In yet another aspect, single nucleotide differences are distinguished through analysis of higher order conformations of single stranded nucleic acids that form in a sequence dependent manner. In this embodiment, single stranded nucleic acids are generated by dissociating the PCR products into single strands, or by preferentially amplifying one strand by using limiting amounts of one primer in the PCR reaction (ie. single-sided PCR). Under selected conditions, the single stranded nucleic acids are allowed to form higher order structures by intramolecular hydrogen bonding of the single stranded nucleic acid. Those skilled in the art are well versed in defining such permissive conditions (i.e. temperature, denaturant concentration, pH, cation concentration etc.) for forming the higher order structures. These conformations, which are sequence dependent and which therefore can be extremely sensitive to single nucleotide changes, affect the electrophoretic mobility of the nucleic acid, and thus reveal variation in a selected nucleotide sequence within a target nucleic acid by their unique electrophoretic mobility profiles. To enhance formation of higher order structures modifications are introduced into the primers used for the PCR reactions. For example, a primer is engineered with additional bases complementary to a part of the selected nucleotide sequence within a target nucleic acid containing the sequence variation, such that higher order conformations form when the additional bases on the primer “snapback” or re-anneal to the normal sequence but not to variant sequences (Wilton, S. D. (1998), Snapback SSCP analysis: engineered conformation changes for the rapid typing of known mutations, Hum. Mutat. 11(3): 252-8). Since the reliability of detecting single nucleotide variations is affected by size of the single stranded probe, conformation analysis using, with each having a distinctive ratio of charge to translational frictional drag, permits detection of a plurality of selected nucleotide sequences within a target nucleic acid while maintaining the optimal length needed for forming higher order structures (Sheffield, V. C., (1993), The sensitivity of single stranded conformation polymorphism analysis for the detection of single base substitutions, Genomics 16 (2): 325-32).

In yet another aspect of the invention, probes are cleaved to detect selected nucleotide sequences within one or more target nucleic acids. In one such embodiment, probes are hybridized to selected nucleotide sequences within one or more target nucleic acids. In another embodiment, PCR products comprising at least one sequence-specific probe serve as substrates for sequence-specific enzymes, such as restriction enzymes. Digestion of the substrates by the enzymes creates cleaved products having a distinctive ratio of charge to translational frictional drag, which provides information about sequence composition of the target polynucleotides. This form of restriction fragment length polymorphism (RFLP) analysis is well known to those skilled in the art (see e.g., Kidd, I. M., (1998), A multiplex PCR assay for the simultaneous detection of human herpesvirus 6 and human herpesvirus 7, with typing of HHV-6 by enzyme cleavage of PCR products, J. Virol. Methods 70 (1): 29-36; Gelernter, J., (1991), Sequence tagged sites (STS) Taq I RFLP at dopamine beta-hydroxylase, Nucleic Acids. Res. 19 (8): 1957).

In another aspect, probes hybridized to selected nucleotide sequences within a target nucleic acid, wherein there is at least one nucleobase not complementary to the corresponding nucleobase in the target nucleic acid, are treated with agents that specifically cleave the non-based-paired nucleotide residues. Generally, the unpaired residue occurs on the hybridized probe (Bhattacharya, et al., (1989), Nucleic Acids. Res. 17, 6821-6840). Although chromosomal DNA may serve as the target nucleic acids, target nucleic acids are cloned DNA fragments comprising selected nucleotide sequences of a target nucleic acid, or PCR amplification products comprising selected nucleotide sequences of a target nucleic acid.

Cleavage may be accomplished with either chemical or enzymatic reagents. In chemical cleavage reactions, the hybrids containing at least one non-complementary nucleobase, are treated with chemicals which specifically modify the unpaired residue, rendering the internucleotide linkage of the modified nucleoside susceptible to hydrolysis. Suitable chemical agents include but are not limited to carbodiimide, osmium tetraoxide, hydroxylamine or potassium permanganate/tetraethylammonium chloride (Ellis, T. P., et al., (1998), Chemical cleavage of mismatch: a new look at an established method, Hum Mutat. 11: 345-53; Roberts, E., (1997), Potassium permanganate and tetraethylammonium chloride are safe and effective substitute for osmium tetraoxide in solid phase fluorescent chemical cleavage mismatch, Nucleic Acids. Res. 25: 3377-78). The use of potassium permanganate/tetraethylammonium chloride rather than osmium tetraoxide enhances cleavage at T/G mismatched pairs.

Enzymatic cleaving reagents encompass a variety of nucleases which recognize unpaired regions. These include but are not limited to single stranded specific nucleases such as S1 nuclease from Aspergillus oryzue, P1 from Penicillum citrinum, and mung bean nuclease (Shenk, et al., (1975) Proc. Natl. Acad. Sci. USA 72 989-93). Although these nucleases are less reactive towards single nucleotide mismatches, they can digest unpaired residues created by longer insertions and deletions (Dodgson, J. B. et al., (1977), Action of single-stranded specific nucleases on model DNA heteroduplexes of defined size and sequence, Biochemistry, 16:2374-49). Cel 1 and SP endonucleases show activity toward unpaired nucleotide residues resulting from nucleotides sequence variations comprising deletions, insertions, and missense mutations, within selected nucleotide sequences of target nucleic acids. (Oleykowski, C. A., (1998) Mutation detection using a novel plant endonuclease, Nucleic Acids. Res. 26: 4597-602; Yeung, A. T., U.S. Pat. No. 5,869,245). Resolvases from various sources, such as bacteriophage and yeast, represent yet another class of cleaving enzymes useful in this embodiment of the invention. Representative examples of resolvases include but are not limited to phage encoded T4 endonuclease VII and T7 endonuclease 1, both of which cleave at mismatches (Cotton, R. G. H., U.S. Pat. No. 5,958,692; Solaro, et al, (1993), Endonuclease VII of Phage T4 Triggers Mismatch Correction in vitro. J. Mol. Biol. 230: 868-877; (Chang, D. Y. et al., (1991), Base mismatch specific endonuclease activity in extracts from Saccharomyces cerevisiae; Nucleic Acids Research 19 (17): 4761-66).

In another aspect, of the present invention encompasses methods that prevent cleavage at unpaired residues. Proteins, including but not limited to the MutS protein of E. coli., bind to sites of single nucleotide mismatches in duplex nucleic acid structures (Su, S. S. et al., (1986), Escherichia coli mutS encoded protein binds to mismatched DNA base pairs, Proc. Natl Acad. Sci. USA 83: 5057-5061). The MutS protein is part of the methylation directed E coli. MutH/S/L mismatch repair system, homologs of which are present in other bacteria, yeast and mammals (Eisen, J. A., (1998), A phylogenetic study of the MutS family of proteins, Nucleic Acids. Res. 26: 4291-300; Alani, E. (1996), The Saccharomyces cerevisiae Msh2 and Msh6 proteins form a complex that specifically binds to duplex oligonucleotides containing mismatched DNA base pairs, Mol. Cell Biol. 16: 5604-15; Modrich, P. et al., (1996), Mismatch repair in replication fidelity, genetic recombination and cancer biology, Annu. Rev. Biochem. 65: 101-33). Therefore in one embodiment of the invention, duplex structures comprising at least one non-base-paired nucleobase unit formed by hybridization of a sequence-specific probe with a selected nucleotide sequence within a target nucleic acid, are treated with mismatch binding proteins such as MutS and then exposed to one or more exonucleases which degrade the duplex strands in a unidirectional fashion. A bound mismatch binding protein inhibits further action of the exonuclease on the strand containing the mismatch, thereby providing nucleic acid products of defined length and which possess a distinctive ratio of charge to translational frictional drag (Ellis, L. A., (1994), MutS binding protects heteroduplex DNA from exonuclease digestion in vitro: a simple method for detecting mutations, Nucleic Acids Res. 22 (13):2710-1; Taylor, G. R., U.S. Pat. No. 5,919,623). Unidirectional exonucleases suitable for use in this assay include, but are not limited to exonuclease III, bacteriophage lambda. exonuclease, and the 3′ to 5′ exonucleases of T7 DNA polymerase, T4 DNA polymerase, and Vent (tm) DNA polymerase.

In yet another aspect, a sequence-specific probe is used in a cleavage based method of detecting selected nucleotide sequences within a target nucleic acid may be a DNA-RNA-DNA probe, where an internal RNA segment is flanked by DNA segments. This tripartite sequence-specific probe is hybridized to a selected nucleotide sequence within a target nucleic acid at a temperature below the Tm of the overall, i.e. tripartite probe. Digestion of this duplex structure with an appropriate RNase, hydrolyzes only the RNA portion of the DNA-RNA-DNA probe when hybridized to a DNA template. In one embodiment, the RNase is a thermo-stable RNase H (Bekkaoui, F., (1996), Cycling probe technology with RNase H attached to an oligonucleotide, Biotechniques, 20 (2): 240-8). If the temperature of the reaction maintained above the Tm of the flanking DNA segments remaining after digestion of the internal RNA segment, those DNA segments dissociate, thus allowing another DNA-RNA-DNA oligomer to associate with the target polynucleotide. Repeated hybridization, RNA cleavage, and dissociation of the flanking DNA segments amplifies the level of detectable dissociated DNA segments. The reaction temperature, in one embodiment, is held constant during the amplification process, thus obviating any need for thermal cycling (Duck, P. (1990), Probe amplifier system based on chimeric cycling oligonucleotides, Biotechniques 9: 142-48; Modrusan, Z. (1998) Spermine-mediated improvement of cycling probe reaction, Mol. Cell Probes 12: 107-16). In this aspect of the present invention, the sequence-specific DNA-RNA-DNA probe used comprise at least one mobility-modifying polymer and at least one reporter molecule attached to either or both of the flanking DNA segments, thereby providing a labeled digestion product having a distinctive ratio of charge to translational frictional drag.

In another aspect, detection of selected nucleotide sequences within one or more target nucleic acids based on cleavage of a probe relies upon cleavage substrates formed by invasive hybridization, as described in Brow et al. U.S. Pat. No. 5,846,717. In this embodiment, the 5′-portion of a sequence-specific probe, which comprises a reporter molecule and which is hybridized to a target nucleic acid, is displaced by a second probe that hybridizes to the same region and thereby exposing that displaced sequence to cleavage with a cleaving reagent. In practicing the embodiment, the target nucleic acid is contacted with a sequence-specific probe and with a second probe. The sequence-specific probe has a 5′-segment complementary to a second region of the selected nucleotide sequence contained within a target nucleic acid and a 3′-portion complementary to a third region of the selected nucleotide sequence contained within a target nucleic acid, wherein the second region is downstream from the third region. The second probe has a 5′-segment complementary to a first region of the selected nucleotide sequence contained within a target nucleic acid and a 3′-segment complementary to the second region of the selected nucleotide sequence contained within a target nucleic acid, wherein the first region is downstream from the second region. Under selected conditions, hybrids form in which the sequenced specific probe and the second probe hybridize to the target polynucleotide such that the second probe displaces the 5′ portion of the hybridized sequence-specific probe, whereas the 3′ portion of the sequence-specific probe and the 5′ portion of the second probe remain annealed to the selected nucleotide sequence contained within a target nucleic acid. The displaced strand, which is a single stranded segment that is not base-paired corresponds to the 5′-end of the sequence-specific probe then serves as a substrate for cleavage nucleases, thus producing discrete digestion products having distinct ratios of charge to translational frictional drag that reflect presence of specific sequences on the target polynucleotide.

Cleaving enzymes recognizing displaced strands are either naturally occurring nucleases or modified nucleases. Naturally occurring structure-specific nucleases include, but are not limited to Pyrococcus woesii FEN-1 endonuclease, thermostable Methoanococcus jannaschii FEN-1 endonucleases, yeast Rad2, and yeast Rad1/Rad10 complex (Kaiser et al., U.S. Pat. No. 6,090,606, Cleavage Reagents; Kaiser, et al. U.S. Pat. No. 5,843,669, Cleavage of nucleic acid using thermostable Methoanococcus jannaschii FEN-1 endonucleases). Other structure-specific enzymes suitable for the cleaving reaction are those derived from modifications of known nucleases and polymerases (Dahlberg et al., U.S. Pat. No. 5,795,763, Synthesis Deficient Thermostable DNA Polymerase; Dahlberg et al., U.S. Pat. No.6,614,402, 5′ Nucleases Derived from Thermostable DNA Polymerase). Modified polymerase that lack polymerase activity but still retain 5′-nuclease activity, are also used as cleaving reagents.

Another embodiment is directed toward the use of the mobility-modifyfing polymers of the present invention in “invader assays,” which are SNP-identifying procedures based upon flap endonuclease cleavage of structures formed by two overlapping probes that hybridize to a target nucleic acid (see e.g. Cooksey et al., 2000, Antimicrobial Agents and Chemotherapy 44: 1296-1301). Such cleavage reactions release products corresponding to the 5′-terminal nucleobase(s) of the “downstream” probe. Where those cleavage products are labeled and can be separated from the uncleaved probe, an invader assay can be used to discriminate single base differences in, for example, genomic sequences or PCR-amplified genomic sequences.

Attachment of the mobility-modifying polymers of the present invention to the labeled 5′-terminus of the downstream probe used in an invader assay provides detectably-labeled cleavage products with distinctive charge to translational frictional drag ratios. Accordingly, a plurality of SNP's are analyzed simultaneously using a plurality of sequence-specific downstream probes, wherein the sequence-specific downstream probes comprise a mobility-modifying polymer of the present invention attached to the labeled 5′-terminus, such that the labeled product generated by flap endonuclease cleavage at each SNP has a distinctive charge to translational frictional drag ratio.

In a further aspect of the invader assay, for example, the downstream probe, which carries a label and a first mobility-modifying polymer of the present invention attached to the 5′-terminus, further comprises a second mobility-modifying polymer attached to the 3′-terminus. The presence of the second mobility-modifying polymer increases the sensitivity of the invader assay by enhancing the difference between the electrophoretic mobility of the flap endonuclease generated product, comprising the 5′-terminus, label, and first mobility-modifying polymer, and the electrophoretic mobility of the uncleaved downstream probe. Accordingly, the second mobility-modifying polymer has a molecular weight of at least 2000. In other embodiments, the second mobility-modifying polymer has a molecular weight of at least 5,000, at least 10,000, at least 20,000, and at least 100,000. In one embodiment, the second mobility-modifying polymer is a mobility-modifying polymer of the present invention, while in other embodiments, the second mobility-modifying polymer is a mobility-modifying polymer of the art, which is, in one illustrative, non-limiting example, an uncharged mono methyl polyethyleneglycol polymer. Moreover, the second mobility-modifying polymer may comprise a mixture of species of different molecular weight, provided that those species do not interfere substantially with detection of the signal product, i.e., the flap endonuclease generated product, comprising the 5′-terminus, label, and first mobility-modifying polymer (see Example 5, below).

More generally, in other embodiments of the present invention, invader assays are performed in which the downstream oligonucleobase polymer comprises a label and a mobility-modifying polymer of the present invention attached to a first region of the downstream oligonucleobase polymer, and a second, high-molecular weight mobility-modifying polymer attached to a second region of the downstream oligonucleobase polymer, wherein first and second regions are separated by the flap endonuclease cleavage site. One aspect of this embodiment is described above and in Example 5, wherein the label and mobility-modifying polymer of the present invention are attached to the 5′-end of the sequence-specific oligonucleobase polymer and a second, high molecular weight mobility-modifying polymer is attached to the 3′-end of the sequence-specific oligonucleobase polymer. In other embodiments, for example, a second, high molecular weight mobility-modifying polymer is attached, via a linker arm nucleotide residue, to the sequence-specific probe, rather than at the 5′-end or 3′-end of the sequence-specific probe. Accordingly, the second, high molecular weight mobility-modifying polymer, is attached at any nucleobase residue within the second region of the downstream probe, or to the 5′-end or 3′-end, whichever is included within the second region of the downstream oligonucleobase polymer. Similarly, in some embodiments, the label, which is a fluorescent dye in certain non-limiting examples, is also attached via a linker arm nucleotide residue at any nucleobase residue within the first region of the downstream probe. Synthesis of such linker arm nucleotides and the coupling of, inter alia, a fluorescent dye or an uncharged mono methyl polyethyleneglycol polymer to the linker, are within the scope of the art (see e.g., Section 4.5 above). Moreover, e.g., linker arm nucleoside phosphoramidite monomers, as well as linker arm nucleoside phosphoramidite monomers comprising flourescent moieties, are commercially available (Glen Research, Inc., Sterling, Va.). In these embodiments, the mobility-modifying polymer of the present invention is attached to the first region of the downstream probe, where the point of attachment may be at the 5′-end or the 3′-end, whichever is encompassed within the first region of the downstream probe, or the mobility-modifying polymer of the present invention may be incorporated within the first region of the downstream probe, providing a molecule according to Structural formula (IV). Therefore, in each of these embodiments, the presence of the second high molecular weight mobility-modifying polymer attached to the second region of the downstream probe increases the sensitivity of the invader assay by enhancing the difference between the electrophoretic mobility of the flap endonuclease generated product comprising a label and a mobility-modifying polymer of the present invention, i.e., the first region of the downstream oligonucleobase polymer, and the electrophoretic mobility of the uncleaved downstream probe.

In a still further embodiment of an invader assay, the downstream probe carries a label and a first mobility-modifying polymer, which is in one non-limiting embodiment, a standard PEO mobility-modifying polymer of the art, that is attached to the first region of the downstream probe, and a second, high molecular weight mobility-modifying polymer attached to the second region of the downstream probe. As above, the presence of the second mobility-modifying polymer increases the sensitivity of the invader assay by enhancing the difference between the electrophoretic mobility of the flap endonuclease generated product, i.e., the first region of the donwstream probe, which comprises a label and a first mobility-modifying polymer, and the electrophoretic mobility of the uncleaved downstream probe. Accordingly, the second mobility-modifying polymer has a molecular weight of at least 2000. In other embodiments, the second mobility-modifying polymer has a molecular weight of at least 5,000, at least 10,000, at least 20,000, and at least 100,000. In one embodiment, the second mobility-modifying polymer is a mobility-modifying polymer of the present invention, while in other embodiments, the second mobility-modifying polymer is a mobility-modifying polymer of the art, which is, in one illustrative, non-limiting example, an uncharged mono methyl polyethyleneglycol polymer.

In another aspect of the present invention, the sequence-specific probe serves as a cleavage substrate in detection reactions involving multiple sequential cleavage reactions, as described in Hall, J. G. et al., U.S. Pat. No. 5,994,069. In this embodiment, a first cleavage structure is formed as set forth above, except that in the present embodiment, the first probe is optionally a sequence-specific probe. The reaction mixture further includes a second target nucleic acid and a third probe, which is a sequence-specific probe, and further comprises at least one attached reporter molecule. The second target polynucleotide has a first, a second and a third region, wherein the first region is downstream of the second region, and the second region is downstream of the third region. The third probe has a 5′ portion fully complementary to the second region of the second target polynucleotide and a 3′ portion fully complementary to the third region of the second target polynucleotide. Treatment of the first cleavage structure results in release of a fourth probe, which has a 5′ portion complementary to the first region of the second target polynucleotide and a 3′ portion fully complementary to the second region of the second target polynucleotide. This released fourth probe forms a cleavage structure with the second target polynucleotide and the third probe under conditions where the 3′ portion of the third probe and the 5′ portion of the fourth probe remains annealed to the second target polynucleotide. Cleavage of the third probe with a cleavage reagent generates a fifth and sixth probe, either or both of which comprise a reporter molecule and a mobility-modifying polymer, thereby providing a digestion product having a distinctive ratio of charge to translational frictional drag. The fifth probe is released upon cleavage, while the sixth probe remains hybridized to the second target polynucleotide until dissociated by denaturation. Subsequent separation and detection of the fifth or sixth probe provides information about the presence of the first and second selected nucleotide sequence within the target nucleic acid.

In a further aspect of the present invention relating to a nucleotide sequence detection method involving multiple sequential cleavage reactions, a first cleavage structure is formed by first and second probe and a selected nucleotide sequence within a target nucleic acid, as set forth above. This aspect of the method further comprises a sequence-specific second target probe, which has a first, a second, and a third region, wherein the first region is downstream of the second region, and wherein the third region upstream of the second region, is fully self complementary and also complementary to the second region, such that it forms a hairpin structure under selected conditions. Cleavage of the first cleavage structure with a cleaving reagent generates a fourth probe, which has a 5′-portion complementary to the first region and a 3′-portion fully complementary to the second region of the probe. Hybridization of the released fourth probe to the first and second regions of the sequence-specific probe forms a second cleavage structure with a displaced third region that is complementary to the second region. Cleavage of this second cleavage structure generates a fifth and sixth probes, either of which comprises a mobility-modifying polymer and a label, thereby providing s digestion product having a distinctive ratio of charge to translational frictional drag, and whose separation and detection provides information about the presence of the first target nucleic acid and the second probe.

Methods for labeling and detecting the cleaved probes, as set forth infra., are equally applicable to the labeling and detection of products of the cleavage reactions. Moreover, labeling of released cleavage products is also accomplished by extension of the product by template independent polymerases, including but not limited to terminal transferase and polyA polymerase as described in U.S. Pat. No. 6,090,606, which is hereby specifically incorporated by reference.

In yet another aspect, the probes of the present invention are employed within a general method to effect the electrophoretic analysis and/or separation of target nucleic acids of identical or different sizes in non-sieving media. Normally, nucleic acids of different length (i.e. consisting of different numbers of nucleobase residues) display an essentially invariant ratio of charge to translational frictional drag. Accordingly, such molecules cannot be separated electrophoretically in non-sieving media. However, attachment of a sequence-specific probe of the present invention to target nucleic acids of identical or different length alters their ratio of charge to translational frictional drag of the target nucleic acids in a manner and to a degree sufficient to effect their electrophoretic mobility and separation in non-sieving media. Furthermore, and in contrast to electrophoretic separations in sieving media, longer nucleic acids to which a sequence-specific probe of the present invention has been attached will migrate more rapidly than a shorter nucleic acid to which the same sequence-specific probe has been attached. Applicants believe, although without wishing to be held to that belief, that such separations are based upon the proportionately smaller effect of attachment of a mobility-modifying sequence-specific probe of defined mass and size to a longer chain nucleic acid molecule than to a shorter chain nucleic acid molecule. Consequently, the ratio of charge to frictional translational drag will be greater for the longer chain, providing the longer chain nucleic acid with a greater velocity in an electric field.

In yet a further aspect, to affect the electrophoretic analysis and/or separation of target nucleic acids sieving media can be employed. Attachment of a sequence-specific probe of the present invention to target nucleic acids of identical or different length alters their ratio of charge to translational frictional drag of the target nucleic acids in a manner and to a degree sufficient to effect their electrophoretic mobility and separation in sieving media providing additional emphasis on the size of the probe -target complex over any net charge affects of the association.

Attachment of a to a population of nucleic acids of different length can be accomplished using a variety of approaches, including but not limited to hybridization, crosslinking of hybridization complexes, enzymatic ligation or direct, synthetic incorporation of the mobility-modifying of the present invention into the population of nucleic acids of different lengths that are to be separated.

In one aspect of this method, a mobility-modifying sequence-specific probe is enzymatically ligated to a population of nucleic acids of different length but having a common nucleotide sequence at the 5′-end, as is seen within the products of a chain termination nucleic acid sequencing reaction or, effectively, in chemical cleavage sequencing reactions which are transparent to all sequences other than those comprising the labeled 5′-end of the nucleic acid substrate. In this embodiment a synthetic template oligonucleotide, having two distinct sequence regions would be used as a template to align the hybridized 3′-end of a mobility-modifying sequence-specific probe so that it would directly abut the hybridized 5′-end, which is generally phosphorylated, that is common to the population of nucleic acids to be separated, and permit the two molecules to be covalently joined. Therefore the 5′-region of the synthetic template oligonucleotide would consist of a nucleotide sequence complementary to the common 5′-end sequence of the molecules to be separated, while the 3′-region of the synthetic template would consist of sequences complementary to the 3′-end of the mobility-modifying sequence-specific probe to be joined. In another embodiment of this approach, the common 5′-end of the population of nucleic acids to be separated corresponds to that generated by a sequence-specific restriction endonuclease. Therefore the synthetic template nucleic acid consists of at least eight nucleobases, of which at least three would be complementary to a common 5′-sequence of the population of molecules to be separated. The design of such template nucleic acids, as well as the conditions under which the enzymatic joining of the hybridized target nucleic acid and the sequence-specific probe would be carried out, are well known to those of ordinary skill in the art. Accordingly, this embodiment of the invention is applicable to any population of molecules of different sizes, provided each has a common 5′-end sequence of at least three nucleotides, in certain embodiments, at least four nucleotides, and in further embodiments, at least eight nucleotides. Similar procedures, wherein the sequence common to a population of molecules of different sizes occurs at the 3′-end, and consequently, the mobility-modifying sequence-specific nucleic to be attached has a phosphorylated 5′-end with the mobility-modifying polymer attached to the 3′-end, are also included within the scope of the present invention.

In a further embodiment, a mobility-modifying sequence-specific probe is synthesized or produced so as to be complementary to a nucleotide sequence within, for example, a sequencing vector, that is upstream of, i.e. toward the 5′-end of, the binding site of a sequencing primer used in Sanger, enzymatic chain termination sequencing reaction. In this embodiment, the sequence-specific probe is enzymatically ligated to the sequencing primer either before or after extension of the sequencing primer during a chain termination sequencing reaction. In this embodiment, the sequence-specific nucleic acid is synthesized so that, once hybridized to the template polynucleotide, its 3′-end would either directly abut the 5′-end of the hybridized sequencing primer, or that 3′-end would hybridize to sequences upstream of the 5′-end of the sequencing primer. In the latter instance, the resulting gap is filled with a nucleic acid polymerase and the extended molecule is then enzymatically ligated to the sequencing primer.

Another embodiment of the invention is related to the separation and/or analysis of probe and polynucleotides. Separation and/or analysis of oligonucleotides is effected by electrophoresis, chromatography, or mass spectroscopy. In methods employing electrophoresis, the format may be thin flat chambers. In another embodiment, the separation and/or analysis is carried out by electrophoresis in capillary tubes. In another embodiment, the separation and/or analysis is carried out by molecular electrophoresis. The advantages of capillary electrophoresis and/or molecular electrophoresis are efficient heat dissipation, which increases resolution and permits rapid separation under high electrical fields. Moreover, the small diameters and/or areas of the capillary tubes or other configurations used for molecular electrophoresis allow separation of numerous samples in arrays of capillaries.

Sieving or nonsieving media are applicable to separation of probes including but not limited to the reaction products generated in the detection methods disclosed herein. Sieving media include covalently crosslinked matrices, such as polyacrylamide crosslinked with bis-acrylamide (see e.g. Cohen, A. S. et al. (1988) Rapid separation and purification of oligonucleotides by high performance capillary gel electrophoresis, Proc. Natl Acad. Sci USA 85: 9660; Swerdlow, H. et al., (1990), Capillary gel electrophoresis for rapid, high resolution DNA sequencing, Nuc. Acids Res. 18 (6): 1415-1419) or linear polymers, for example hydroxypropylmethylcellulose, methyl cellulose, or hydroxylethylcellulose (Zhu et al. (1992), J. Chromatogr. 480: 311-319; Nathakarnkitkool, S., et al. (1992), Electrophoresis 13: 18-31).

In one embodiment, the electrophoretic medium is a non-sieving medium. Although polynucleotides are not readily separable in a non-sieving medium, probes and polynucleotides have distinctive ratios of charge to translational frictional drag that permit separation in a non-sieving media, even when the probe and polynucleotides are of the same length.

### EXAMPLES

Without further elaboration, it is believed that one skilled in the art can, using the preceding description, utilize the present invention to its fullest extent. The following specific examples are offered by way of illustration and not by way of limiting the remaining disclosure. Two hybridization probes were made with sequence specific recognition sequences for PhiX174 DNA. In addition to the specific recognition sequences, each probe has a portion of double stranded DNA to which either fluorescent tags or biotin were added.

The probes were made using the following protocol.

LNA-DNA Chimera Sense Strand Primers

(SEQ ID NO:1)

5′ gct cag gaa caa aga aac gcA GGG AGA GAG GAA GGA
at tca cca gtc aca cga cca

(SEQ ID NO:2)

5′ cag taa cag ata caa act caA GGG AGA GAG GAA GGA
at tca cca gtc aca cga cca

Blocking Oligo

| | |
|---|---|
| 5′ CTCCTTCCTCTCTCCCT | (SEQ ID NO:3) |

DNA Antisense Primer

| | |
|---|---|
| 5′ ggtgctgctatcgatggttt | (SEQ ID NO:4) |

UPPER CASE=LNA, lower case=DNA

Annealing LNA-DNA chimera with Blocker LNA oligo

The LNA-DNA chimera and LNA blocker oligo were mixed in 10 mM Tris-HCl (pH 7.4), 0.1 mM EDTA, overlaided with mineral oil, and annealed by heating the mixture to 105° C. for 2 minutes and slowly cooling to 25° C. over 45 minutes in an MJ Research PTC-200 DNA Engine.

Reaction mixtures containing 0.1□M annealed LNA-DNA chimera/Blocker hetero-dimer, 0.1 □M antisense primer, 1× NovaTaq™ Buffer+MgCl₂, 0.2 mM dGTP, 0.2 mM dCTP, 0.2 mM dATP, 50□M dTTP, 0.3 mM Amino-allyl-dUTP, 4% Dimethylsulfoxide, 35 pM double stranded M13mpl18 RF I DNA, and 0.05 Units/□L NovaTaq™ DNA Polymerase were prepared to a final volume of 100 □L in a 0.5 mL tube adapted for a thermal cycling reaction. In addition, one reaction contained 0.07 mM biotin dUTP. The reactions were thermocycled as follows: denaturation at 95° C. for 2 min, followed by repeated cycles of: 95° C. for 15 sec and 65-66° C. for 4.5 min, for 30 cycles; 74° C. for 4 min, 4° C. hold cycle.

The reactions were purified over a spin column. The product was eluted in 11 □L of nuclease-free water. Fluorescent dye was coupled to the probe that did not have biotin dUTP incorporated. In summary, two probes were made with recognition sequences to PhiX174. One probe is labeled with fluorescent dye and the other probe has incorporated biotin dUTP and no fluorescent dye.

Two hybridization reactions were carried out in 2×SSC buffer at 37° C. for 2 hours. Hybridization 1 contained 1 pM labeled probe, 1 pM CM probe, and 0.5 pM target PhiX174 DNA. Hybridization 2 contained 1 pM labeled probe, 1 pM CM probe, and no target PhiX174 DNA. After the 2 hour hybridization, 5□l of each reaction was incubated with streptavidin nanospheres. Samples were diluted to 25 fM each probe in electrophoresis buffer and were subjected to 0.03 mAmps of current for 8 minutes in the single molecule electrophoresis instrument.

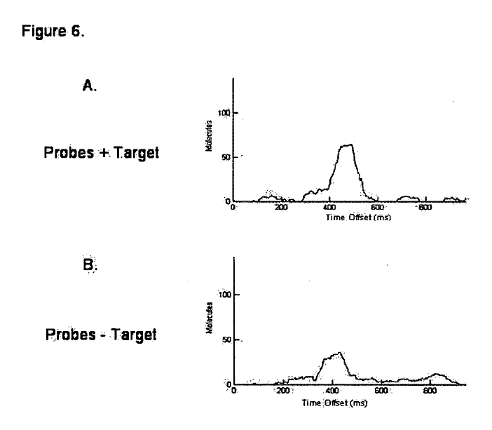

Adjacent photon bursts were grouped into photon bursts derived from molecules using the instrument software. Molecule-derived photon bursts were then crosscorrelated to determine their electrophoretic velocities (time offset for detection at the two detectors). The histogram plots of the molecule crosscorrelations are shown in FIGS. 6a and 6b. With labeled probe and biotin-streptavidin-nanosphere (CM) probe alone, a histogram peak is seen at a velocity (elapsed time) of 400 msec. This corresponds to the dye-labeled probe. When the probes are hybridized to target, a new peak is seen at 485 msec which corresponds to the hybrid formed between the two probes and the target nucleic acid. The hybrid has an altered charge to mass ratio and a distinct velocity as a result of hybridization with the mobility modified (CM) probe

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### FRERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

1. A method for detecting molecules, molecular interactions or molecular complexes comprising:
(a) detecting, through interaction with at least one probe, an interaction between the probe and a molecule, wherein at least one probe has a differentiating physical characteristic selected from the group consisting of a net charge, a mass, and any combination thereof, and at least one probe has a detectable parameter; and
(b) differentiating between an unbound probe or probes of (a) and a probe or probes bound to the molecule.

2. A method according to claim 1, wherein the at least one probe interacts with a feature of the molecule selected from the group consisting of a site on a target molecule species, a probe attached to the molecule, and any combination thereof.

3. A method according to claim 1, wherein the differentiating physical characteristic between the probe and molecule is differentiable from one or more of the other probes, when two or more probes interact with the molecule.

4. A method according to claim 1, wherein the differentiation of the probe is based upon the behavior of both the unbound CM probe and the bound CM probe target complex, or the behavior of either the unbound CM probe or the bound CM probe-target complex by virtue of the detected entity's or entities' behavior in an electric field.

5. A method according to claim 1, wherein one or more of the detectable probes also is a CM probe

6. A method according to claim 1, wherein either the target or the CM probe-target complex is a detectable.

7. A method according to claim 1, wherein both the target and the CM probe-target complex are detectable.

8. A method according to claim 1, wherein the probe or probes is selected from among a nucleic acid, oligonucleotide, PNA, LNA, XLNT, peptide, polypeptide, lipid, sterol, biological molecule, dye, drug, small molecule, mass tag, isotope, microsphere, nanosphere, barcode particle, or nanocrystal, and any combination thereof.

9. A method according to claim 1, wherein at least one of the detectable parameters is chosen from among luminescence, chemiluminescence, light absorption, light scattering, fluorescence, fluorescence lifetime, fluorescence polarization, emission burst size, emission burst length, emission wavelength, emission intensity, electrical reactance, enzyme activity, diffusion, cooperative hybridization, EPR, SER, SPR, raman emission, microwave, electrophoretic velocity, electrokinetic velocity and other luminescent or non-luminescent-related parameters known to those experienced in the art.

10. A method according to claim 1, wherein at least one of the probes is labeled with one or more dye molecules

11. A method according to claim 1, wherein the detectable parameter is measured by optical or electrical detection

12. A method according to claim 11, wherein two or more detectors are used

13. A method according to claim 11, wherein one or more detectors are chosen from among avalanche photodiodes, and a CCD camera

14. A method according to claim 13, wherein the CCD camera serves to detect two or more parameters

15. A method according to claim 13, wherein the CCD camera detects one or more parameters from two or more positions within the sample

16. A method according to claim 1, wherein detection uses spatial or temporal measurements for detection

17. A method according to claim 1, wherein two or more nucleic acid probes are used

18. A method according to claim 17, wherein one or more of the probes contains one or more CM tags

19. A method according to claim 17, wherein one or more of the probes is labeled with fluorescent dye molecules

20. A method according to claim 19, wherein the probe and probe complexes are detected at more than one detector.

21. A method according to claim 20, wherein the data from two of more detectors is cross-correlated

22. A method according to claim 20, wherein two or more of the detectors are spatially separated.

23. A method according to claim 22, wherein correlated parameter data is used to determine electrophoretic velocities of detected entities.

24. A method according to claim 23, wherein electrophoretic velocity is one of the parameters used to differentiate between molecular species.

25. A method according to claim 23, wherein fluorescence measurements are used to detect the target, one or more probes, and/or the complex formed as a result of interaction of the target with one or more probes.

26. A method according to claim 1, wherein multiple targets are detected in a single assay.

27. A method according to claim 1, wherein unbound probes are removed from the sample prior to analysis

28. A method according to claim 1, wherein unbound probes are made undetectable prior to analysis.

29. A method according to claim 27, wherein one or more probes is released from the probe-target complex after specific interaction of the target with one or more probes.

30. A method according to claim 29, wherein the released probes are detected and serve as a measure of the number of probe-target complexes present.

31. A method according to claim 27, wherein probes remaining on the target are detected and these serve as a measure of the target present.

32. A method according to claim 1, wherein one or more of the probes is bound to a surface

33. A method according to claim 1, wherein one or more of the probes is bound to a particle.

34. A method according to claim 1, wherein the sample is in solution.

35. A method according to claim 1, wherein the sample also contains a sieving matrix

36. A method for detecting and molecules, molecular interactions or molecular complexes, consisting of:
a. One or more probes
b. Each probe recognizing (via hybridization, binding, etc.) at least one target molecule species, or site on a target molecule species (or another probe), and
c. At least one of the probes has a net charge, mass or net charge and mass (any or the three or which is designated as a CM tag) that is differentiable from the target or from one or more of the other probes, and
d. At least one probe or target that has a detectable parameter
e. Differentiating between an unbound CM probe and a probe bound to a target (complex, forming an interaction, or to another probe) based upon a temporal difference in detection between the unbound CM probe and the bound CM probe target complex in an electric field, or
f. Differentiating CM probe based upon a temporal difference in detection of either the unbound CM probe or the bound CM probe-target complex by virtue of temporal difference in detection between the detected entity's or entities' behavior in an electric field.

37. The method of claim 36 where one or more of the detectable probes also is a CM probe

38. The method of claim 36 where either the target or the CM probe-target complex is a detectable.

39. The method of claim 36 where both the target and the CM probe-target complex are detectable.

40. The method of claim 36 where the probe or probes is selected from among a nucleic acid, oligonucleotide, PNA, LNA, XLNT, peptide, polypeptide, lipid, sterol, biological molecule, dye, drug, small molecule, mass tag, isotope, microsphere, nanosphere, barcode particle, or nanocrystal, and any combination thereof.

41. The method of claim 36 where at least one of the detectable parameters is chosen from among luminescence, chemiluminescence, light absorption, light scattering, fluorescence, fluorescence lifetime, fluorescence polarization, emission burst size, emission burst length, emission wavelength, emission intensity, electrical reactance, enzyme activity, diffusion, cooperative hybridization, EPR, SER, SPR, raman emission, microwave, electrophoretic velocity, electrokinetic velocity and other luminescent or non-luminescent-related parameters known to those experienced in the art.

42. The method of claim 33 where at least one of the probes is labeled with one or more dye molecules

43. The method of claim 33 where the detectable parameter is measured by optical or electrical detection

41. The method of claim 40 where two or more detectors are used

42. The method of claim 40 where one or more detectors are chosen from among avalanche photodiodes, and a CCD camera

43. The method of claim 42 where the CCD camera serves to detect two or more parameters

44. The method of claim 42 where the CCD camera detects one or more parameters from two or more positions within the sample

45. The method of claim 33 where detection uses spatial or temporal measurements for detection

46. The method of claim 33 where two or more nucleic acid probes are used

47. The method of claim 46 where one or more of the probes contains one or more CM tags

48. The method of claim 46 where one or more of the probes is labeled with fluorescent dye molecules

49. The method of claim 16 where the probe and probe complexes are detected at more than one detector.

50. The method of claim 49 where the data from two of more detectors is cross-correlated

51. The method of claim 49 where two or more of the detectors are spatially separated.

52. The method of claim 51 where correlated parameter data is used to determine electrophoretic velocities of detected entities.

53. The method of claim 52 where electrophoretic velocity is one of the parameters used to differentiate between molecular species.

54. The method of claim 52 where fluorescence measurements are used to detect the target, one or more probes, and/or the complex formed as a result of interaction of the target with one or more probes.

55. The method of claim 33 where multiple targets are detected in a single assay.

56. The method of claim 33 where unbound probes are removed from the sample prior to analysis

57. The method of claim 33 where unbound probes are made undetectable prior to analysis.

58. The method of claim 57 where one or more probes is released from the probe-target complex after specific interaction of the target with one or more probes.

59. The method of claim 57 where the released probes are detected and serve as a measure of the number of probe-target complexes present.

60. The method of claim 58 where probes remaining on the target are detected and these serve as a measure of the target present.

61. The method of claim 33 where one or more of the probes is bound to a surface

62. The method of claim 33 where one or more of the probes is bound to a particle.

63. The method of claim 33 where the sample is in solution.

64. The method of claim 33 where the sample also contains a sieving matrix
